SEQUENCE LISTING (4 sequences)

SEQ ID NO: 1 - protein, 8 aa, Artificial Sequence
  bradykinin-related polypeptides
HWESASXX

SEQ ID NO: 2 - protein, 10 aa, Artificial Sequence
  the bradykinin-related polypeptides
XHWESASXXR

SEQ ID NO: 3 - protein, 9 aa, Artificial Sequence
  the bradykinin-related polypeptides
HWESASLLR

SEQ ID NO: 4 - protein, 5 aa, Artificial Sequence
  bradykinin-related peptides
HXGXA

(19) United States
(12) Patent Application Publication
(10) Pub. No.: US 2014/0221236 A1
(43) Pub. Date: Aug. 7, 2014

(54) METABOLOMIC MARKERS FOR PRETERM BIRTH

(71) Applicants: Kristi S. Borowski, Byron, MN (US); Jeff Murray, Iowa City, IA (US); Kelli K. Ryckman, North Liberty, IA (US)

(72) Inventors: Kristi S. Borowski, Byron, MN (US); Jeff Murray, Iowa City, IA (US); Kelli K. Ryckman, North Liberty, IA (US)

(21) Appl. No.: 14/171,550

(22) Filed: Feb. 3, 2014

Related U.S. Application Data
(60) Provisional application No. 61/759,939, filed on Feb. 1, 2013.

(51) Int. Cl. G01N 33/68; G01N 33/92; G01N 33/49

(52) U.S. Cl. CPC G01N 33/689; G01N 33/492; G01N 33/92
USPC 506/9; 506/15; 506/18; 506/12

(57) ABSTRACT

Panels, kits, and methods for diagnosing or predicting the likelihood of occurrence of preterm birth (PTB) in a subject are disclosed. An exemplary panel includes at least one of a first marker from a first group of markers and a second marker from a second group of markers. A significant decrease in the first marker and a significant increase in the second marker measured in a sample taken from a subject during the second trimester of pregnancy are diagnostic or predictive of an increased risk of preterm birth for the subject.

### CROSS REFERENCE TO RELATED APPLICATIONS

This application claims the benefit of U.S. Application Ser. No. 61/759,939, filed Feb. 1, 2013, which is incorporated herein by reference.

### REFERENCE REGARDING FEDERALLY SPONSORED RESEARCH OR DEVELOPMENT

This invention was made with government support under HD 052953 and HD 057192 awarded by the National Institutes of Health. The Government has certain rights in the invention.

### BACKGROUND OF THE INVENTION

Preterm birth (PTB) is the leading cause of morbidity and mortality for newborns both worldwide and in the United States [1]. Many risk factors are known to be related to PTB, but identifying at risk pregnancies before the onset of labor has proven to be difficult [2]. Maternal obstetric history of past PTB is, to date, the most easily implemented and widely used screening measure. Cervical length measurement is also beneficial, though not currently universally implemented, despite evidence for its use in all singleton pregnancies [3,4]. Interventions in response to both short cervical length and past PTB have proven to be effective [5,6]. Many other screening measures and biomarkers have been proposed but none are used ubiquitously or justify additional testing, especially in low-risk individuals [7]. Thus, a screening tool based on existing and easily available clinical and laboratory data may be useful.

Cholesterol is a common and inexpensive serum test and has been associated with PTB. A recent report indicated that both high (>90th percentile) and low (<10th percentile) cholesterol levels during the second trimester of pregnancy may identify women at risk for PTB [8]. Each type of abnormal cholesterol measurement conferred an increased odds of PTB by a factor >2.5. A similar finding was subsequently reported using pre-pregnancy lipids indicating a “U-shaped” relationship between cholesterol and PTB [9]. These findings are intriguing given that low-density lipoprotein (LDL) is the precursor to progesterone synthesis during pregnancy [10]. In addition, commonly obtained analytes used in maternal serum screening have yielded similar risk profiles for PTB. Alpha-fetoprotein (AFP) and Inhibin A >2.0 multiples of the median (MoM) each more than double the odds of subsequent PTB [11,12].

To date, only a limited number of biomarkers have demonstrated high specificity, however, they lack sensitivity and positive predictive value making them a poor tool for risk stratification. There is an evident need to stratify women at risk for PTB to offer additional screening, or test possible interventions if appropriate. The present disclosure provides a non-invasive screening tool applicable at the population-level that improves upon obstetric history alone to predict PTB.

### SUMMARY OF THE INVENTION

In the first aspect, a panel for diagnosing or predicting the likelihood of occurrence of preterm birth (PTB) in a subject includes at least one of (a) a first marker from a first group of markers that when significantly lower in concentration compared to a term birth control is predictive of preterm birth, and (b) a second marker from a second group of markers that when significantly higher in concentration compared to the term birth control is predictive of preterm birth. A significantly lower concentration of the first marker and/or a significantly higher concentration of the second marker measured in serum taken from a subject during the second trimester of pregnancy compared to a control are diagnostic or predictive of an increased risk of preterm birth for the subject.

In one embodiment of the invention, the panel includes a marker from the first group and a marker from the second group. In another embodiment, the panel includes at least two markers from the first group and at least two markers from the second group. In a further embodiment, the panel comprises at least three markers from each group.

In one embodiment of the panel, the first group of markers includes at least one of glutamine, serotonin, tryptophan, kynurenine, alpha-tocopherol, beta-tocopherol, gamma-tocopherol, gamma-CEHC, glycolithocholate sulfate, taurolithocholate 3-sulfate, biliverdin, bilirubin (Z,Z), bilirubin (E,E), 3-methyl-2-oxobutyrate, 3-methyl-2-oxovalerate, 4-methyl-2-oxopentanoate, isobutyrylcarnitine, 3-hydroxyisobutyrate, 2-methylbutyrylcarnitine, isovalerylcarnitine, propionylcarnitine, phenyllactate (PLA), phenylacetylglutamine, p-cresol sulfate, indolepropionate (IPA), 7-alpha-hydroxy-3-oxo-4-cholestenoate (7-Hoca), 3-indoxyl sulfate, cholesterol, and serum urea.

In another embodiment, the second group of markers includes at least one of glutamate, anthranilate, 13-HODE, 9-HODE, leukotriene B4, 5-HETE, 5-oxoETE, 9(S)-hydroperoxy-10E,12Z-octadecadienoic acid (9(S)-HpODE), 5-HEPE, methionine sulfoxide, HWESASXX (SEQ ID NO:1), XHWESASXXR (SEQ ID NO:2), HWESASLLR (SEQ ID NO:3), HXGXA (SEQ ID NO:4), prostaglandin A2, prostaglandin E2, 7-alpha-hydroxycholesterol, 7-beta-hydroxycholesterol, a γ-glutamyl amino acid, and arginine.

In a further embodiment, the second group of markers further includes at least one fatty acid selected from the group consisting of caproate, heptanoate, caprylate, and pelargonate.

In another embodiment, the control comprises serum from one or more subjects who experienced term birth.

In a further embodiment, a kit includes at least one panel including a marker from the first group and a marker from the second group, a detection reagent specific for the marker from the first group, and a detection reagent specific for the marker from the second group.

In a further embodiment, the detection reagent includes a chemical compound, an antibody, or other reagent that uniquely interacts with a single marker to enable detection of the marker.

In yet another embodiment, a method of diagnosing or predicting the likelihood of occurrence of preterm birth (PTB) in a subject includes the steps of collecting a serum sample from a subject during the second trimester of pregnancy and measuring differences in levels of markers of the above-mentioned panel compared to a control. The levels of one or more of the markers are measured by means of one or more of antibody detection, chromatography, mass spectrometry, and a lipid test.

In second aspect, a kit for diagnosing or predicting the likelihood of occurrence of preterm birth (PTB) in a subject includes a support, a first marker from a first group of markers immobilized on the support, a second marker from a second group of markers immobilized on the support, and a detection reagent. A significant decrease in the first marker and a significant increase in the second marker measured in serum taken from a subject during the second trimester of pregnancy compared to a control are diagnostic or predictive of an increased risk of preterm birth for the subject.

In one embodiment, the kit includes a marker from the first group and a marker from the second group.

In another embodiment the kit includes at least two markers from the first group and at least two markers from the second group.

In a further embodiment, the kit includes at least three markers from each group.

In another embodiment, the first group of markers includes at least one of glutamine, serotonin, tryptophan, kynurenine, alpha-tocopherol, beta-tocopherol, gamma-tocopherol, gamma-CEHC, glycolithocholate sulfate, taurolithocholate 3-sulfate, biliverdin, bilirubin (Z,Z), bilirubin (E,E), 3-methyl-2-oxobutyrate, 3-methyl-2-oxovalerate, 4-methyl-2-oxopentanoate, isobutyrylcarnitine, 3-hydroxyisobutyrate, 2-methylbutyrylcarnitine, isovalerylcarnitine, propionylcarnitine, phenyllactate (PLA), phenylacetylglutamine, p-cresol sulfate, indolepropionate (IPA), 7-alpha-hydroxy-3-oxo-4-cholestenoate (7-Hoca), 3-indoxyl sulfate, cholesterol, and serum urea.

In a further embodiment, the second group of markers includes at least one of glutamate, anthranilate, 13-HODE, 9-HODE, leukotriene B4, 5-HETE, 5-oxoETE, 9(S)-hydroperoxy-10E,12Z-octadecadienoic acid (9(S)-HpODE), 5-HEPE, methionine sulfoxide, HWESASXX (SEQ ID NO:1), XHWESASXXR (SEQ ID NO:2), HWESASLLR (SEQ ID NO:3), HXGXA (SEQ ID NO:4) prostaglandin A2, prostaglandin E2, 7-alpha-hydroxycholesterol, 7-beta-hydroxycholesterol, a γ-glutamyl amino acid, and arginine. In a third aspect, a method of determining the likelihood of preterm birth in a subject is disclosed. The method includes the steps of analyzing a sample taken from a subject to determine the levels of a first marker that when significantly lower in concentration compared to a term birth control is predictive of preterm birth and a second marker that when significantly higher in concentration compared to a term birth control is predictive of preterm birth, and comparing the levels of the first and second markers to a term birth metabolic profile in order to determine the likelihood of preterm birth in the subject.

In one embodiment, the levels of the first and second markers are measured by means of one or more of antibody detection, chromatography, mass spectrometry, and a lipid test.

In another embodiment, the sample is taken from a subject during the second trimester of pregnancy.

### DESCRIPTION OF THE INVENTION

The present invention is based, at least in part, on the discovery that certain metabolic markers in maternal serum (or other samples) can be used to diagnose or predict the likelihood of occurrence of preterm birth or preterm labor of a pregnant subject. By analyzing a biological sample (e.g., blood, serum, tissue, bodily fluid, and the like) from a subject to determine a level of one or more of these metabolic markers in the sample to assemble a metabolic profile of these markers and comparing the subject's metabolic profile to that of a control sample, one may diagnose or predict the likelihood of preterm birth of the subject. The control sample may either have a metabolic profile consistent with term birth or alternatively a metabolic profile that is consistent with preterm birth. In this way, a subject having a metabolic profile that is essentially same as or similar to that of the term birth metabolic profile may be predicted to have a term birth. Alternatively, a subject with a preterm birth metabolic profile may be predicted to have a preterm birth. Similarly, a subject that has a metabolic profile significantly different than a term birth metabolic profile may also be predicted to have a preterm birth.

The metabolic markers identified that enable diagnosis or prediction of preterm birth include two general categories. The first category or first group includes markers that when analyzed in a subject who will experience preterm birth exhibit lower levels and/or concentrations than those from a term birth metabolic profile.

The second category or second group includes markers that when analyzed in a subject who will experience preterm birth exhibit higher levels and/or concentrations. Therefore, panels of identified metabolic markers may be assembled based on a subset (one or more) of markers from each of the first group and second group of markers to create predictive metabolic profiles when analyzed in a subject sample.

By “preterm birth” or “PTB,” we mean a birth about <37 weeks (for a human) completed gestational age and term birth (TB) was considered about >37 completed weeks gestational age as indicated on the birth certificate.

By “preterm labor,” we mean the onset of labor symptoms at less than 37 weeks gestational age. Labor symptoms include cramps or contractions, watery discharge from the vagina, backache, severe pelvic pressure, and blood from the vagina. Preterm labor may or may not progress into pre-term birth. In one embodiment, PTL means labor that begins on or after 22 weeks gestational age.

By “patient” or “subject,” we mean a female mammalian animal, including a human, a veterinary or farm animal, a domestic animal or pet, and animals normally used for clinical research, including non-human primates, dogs and mice. The definitions for preterm birth for non-human mammals include birth less than about 90% term. More specifically, the subject of these methods is a human. In one aspect of the methods described herein, the subject undergoing the diagnostic or therapeutic method is asymptomatic for pre-term birth. In another aspect, the subject undergoing the diagnostic or therapeutic methods described herein shows clinical symptoms, or history, of preterm birth.

By “likelihood (or increased risk) of preterm birth,” we mean an increase in the risk or probability that the subject will develop preterm birth as compared a defined control population of mammalian subjects who experience full term birth. For example, the control may comprise a single healthy pregnant subject at the time of pregnancy, or a population of multiple healthy pregnant subjects at the time of pregnancy or multiple healthy pregnant subjects who did not develop preterm birth, or a population of multiple healthy pregnant subjects at the time of pregnancy or multiple healthy pregnant subjects who had preterm labor but did not develop preterm birth, or the same subject at an earlier time in the pregnancy, or one or multiple subjects with one or more clinical indicators of PTB, but who did not develop PTB. In addition, a predetermined control may also be a negative predetermined control. For example, a negative predetermined control comprises one or multiple subjects who had PTB. In one embodiment, the present invention discloses a panel for diagnosing or predicting the likelihood of occurrence of preterm birth (PTB) in a subject. The panel may include at least two markers. At least one of the markers in the panel may be chosen from the upregulated group (first group or category). At least one of the markers in the panel may be chosen from a downregulated group of markers (second group or category). In one embodiment, the present panel may comprise at least two markers from each group or three markers from each group, or one from the downregulated and two or more from the upregulated, or vice versa. To be diagnostic or predictive of preterm birth, differences in levels or concentrations may be about 1%, 5%, 10%, 15%, 20%, 25%, 30%, 35%, 40%, 45%, 50%, 55%, 60%, 65%, 70%, 75%, 80%, 85%, 90%, 95%, 100%, 150%, or 200%. Similarly, to be predictive of preterm birth differences in levels or concentrations of metabolic markers in a subject sample compared to a control may be about at least one half fold, one fold, one and half fold, two fold, two and half fold, three fold, three and half fold, four fold, four and half fold, five fold, five and half fold, six fold, six and half fold, seven fold, seven and half fold, eight fold, eight and half fold, nine fold, nine and half, or ten fold. These differences may vary about ±5%.

In another embodiment, it is believed that the risk of preterm birth may be monitored over time by analyzing subject samples and comparing levels of markers to a control. For example, more than one sample may be taken from the subject over time to analyze marker levels. Further, it is envisioned that control profiles assembled from term birth metabolic profiles taken at different time points throughout pregnancy may be used to compare to subject samples at equivalent gestational time periods. For example, subject samples may be taken at least once during each trimester and compared to equivalent time term birth controls to monitor the risk of preterm birth throughout the pregnancy.

In one embodiment, a panel for diagnosing or predicting the likelihood of occurrence of preterm birth (PTB) in a subject includes at least one of (a) a first marker from a first group of markers and (b) a second marker from a second group of markers. A significantly lower level or concentration of the first marker and/or a significantly higher level or concentration of the second marker measured in serum taken from a subject during the second trimester of pregnancy compared to a term birth control is diagnostic or predictive of an increased risk of preterm birth for the subject.

In another embodiment, the panel includes a marker from the first group and a marker from the second group, or at least two markers from the first group and at least two markers from the second group, or at least three markers from each group. It is further envisioned that the panel may include a single marker from one group and a plurality of markers from the other group, such as 2, 3, 4, 5, 6, 7, 8, 9, 10 or more markers. Any number of markers may be included in the panel from either group, as desired.

The first group of markers may include at least one of glutamine, serotonin, tryptophan, kynurenine, alpha-tocopherol, beta-tocopherol, gamma-tocopherol, gamma-CEHC, glycolithocholate sulfate, taurolithocholate 3-sulfate, biliverdin, bilirubin (Z,Z), bilirubin (E,E), 3-methyl-2-oxobutyrate, 3-methyl-2-oxovalerate, 4-methyl-2-oxopentanoate, isobutyrylcarnitine, 3-hydroxyisobutyrate, 2-methylbutyrylcarnitine, isovalerylcarnitine, propionylcarnitine, phenyllactate (PLA), phenylacetylglutamine, p-cresol sulfate, indolepropionate (IPA), 7-alpha-hydroxy-3-oxo-4-cholestenoate (7-Hoca), 3-indoxyl sulfate, cholesterol, and serum urea.

The second group of markers may include at least one of glutamate, anthranilate, 13-HODE, 9-HODE, leukotriene B4, 5-HETE, 5-oxoETE, 9(S)-hydroperoxy-10E,12Z-octadecadienoic acid (9(S)-HpODE), 5-HEPE, methionine sulfoxide, HWESASXX (SEQ ID NO:1), XHWESASXXR (SEQ ID NO:2), HWESASLLR (SEQ ID NO:3), HXGXA (SEQ ID NO:4), prostaglandin A2, prostaglandin E2, 7-alpha-hydroxycholesterol, 7-b eta-hydroxycholesterol, a γ-glutamyl amino acids, and arginine.

In another embodiment, a kit is envisioned that includes a panel as described above. The kit may include any suitable substrate as a support. For example, a support may include a substrate that may be selected from glass, plastic, cellulose, nitrocellulose, a non-woven material, metal, and combinations thereof. The substrate may further be a flat sheet, a plate with multiple wells, a chip, a platform with separate regions, and the like. Additional conventional tests may be included on the substrate or separately from the substrate but included in the same kit as the substrate. The kit may include at least a first marker from a first group of markers and at least a second marker from a second group of markers as discussed above immobilized on the support. The kit may further include any suitable means for detection (immobilized on or similarly associated with or separate from the support), such as detection reagents, and auxiliary materials used for detection, such as buffers, standards, and the like. In one embodiment, the kit may include at least one detection reagent, such as a chemical compound, an antibody, or other reagent that uniquely interacts with a single marker to enable detection of the marker. A suitable detection technique may include chromatography, mass spectrometry, UV-Vis or IR spectroscopy, fluorescence, chromogenic detection, immunofluorescent detection, or other antibody detection assay, such as ELISA.

In one embodiment, the kit may be used, for example, as a standard for chromatography assays, including gas chromatography, high pressure liquid chromatography, column chromatography, and the like in series with or separate from mass spectrometry, for example, single or tandem mass spectrometry, and the like. Additional analyses of the panel to measure and or compare serum levels of the markers within a subject sample are contemplated herein to the extent known in the art.

In a further embodiment, an antibody detection assay specific for detection of one or more of the first group of markers and for one or more of the second group of markers of the panel is envisioned, such as an ELISA. The ELISA may include antibodies specific for antigens or epitopes of the markers of the panel. An antigen can be a natural or synthetic protein or fragment thereof, polysaccharide, or nucleic acid. Skilled artisans know that antigens can induce an immune response and elicit antibody formation. Antibodies can be molecules synthesized in response to the presence of a foreign substance, wherein each antibody has specific affinity for the foreign material that stimulated its synthesis. The specific affinity of an antibody need not be for the entire molecular antigen, but for a particular site on it called the epitope (Kindt et al., Kuby Immunology, 6th Edition 574 pps, (2006), incorporated herein by reference as if set forth in its entirety). Antibodies can be, for example, a natural or synthetic protein or fragment thereof, or nucleic acids (e.g., aptamers) with protein-binding or other antigen-binding characteristics. Antibodies can be produced in response to antigenic stimuli including, but not limited to, exposure to foreign proteins, microorganisms, and toxins. When the panel is contacted with a sample containing at least one antibody specific to an antigen in the panel, an immunocomplex forms between the antigen and the antibody specific for the antigen. One of ordinary skill in the art can assess antigen-antibody immunocomplex formation by techniques commonly used in the art. Examples of suitable additional assays to assess immunocomplex formation contemplated herein include phage immunoblot and radioimmunoassay. See, e.g., (Dubovsky et al., J. Immunother. 30:675-683 (2007), incorporated herein by reference as if set forth in its entirety).

In one embodiment, the present application discloses a method of diagnosing or predicting the likelihood of occurrence of preterm birth (PTB) in a subject by using a panel or a kit as discussed above. The method may include the steps of collecting a sample from a subject during the second trimester of pregnancy, measuring the level of a first marker from a first group of markers and a second marker from a second group of markers, calculating the differences in levels of the first marker and the second marker compared to a control, and determining the likelihood of occurrence of preterm birth (PTB) in the subject. A significant decrease in concentration of the first marker and a significant increase in concentration of the second marker measured in the sample taken from a subject during the second trimester of pregnancy compared to a control are diagnostic or predictive of an increased risk of preterm birth for the subject.

In one specific embodiment, a panel or a kit may include standards for the first and second markers. For example, a first quantity of a first marker at a known concentration and a second quantity of a second marker at a known concentration may be immobilized or otherwise associated with a support substrate. Such a panel or kit may serve as a standard platform for analysis by one or more analytical means to predict or monitor risk of preterm birth. Alternatively or in addition, a sample (e.g., serum, blood, or other bodily fluid or tissue) from a subject may be added to the support substrate, one or more detection reagents may be applied to the support substrate, and the levels of the first marker and the second marker in the sample and those predisposed on the substrate may be measured by a suitable detection technique and compared to determine relative levels of the markers in the sample. The likelihood of occurrence of preterm birth (PTB) in the subject may then be determined based on the results of the comparison.

In another embodiment, a panel or a kit may further include one or more detection reagents. In one embodiment, a detection reagent may exhibit a detectable signal, such as a color, light, heat, and the like in the presence of a specific marker. In one preferred embodiment, the magnitude of the signal may be relative to the level of the marker. In this way, the relative magnitude or intensity of signal of a particular detection reagent as a result of interaction with a specific marker may be used to determine the relative concentration of the marker in the sample. In such a configuration, the kit may further include instructions to read the results of the comparison, and may include a scale to translate the magnitude of the signal to a fold difference in concentration of a marker in order to determine the likelihood of preterm birth in the subject from whom the sample was taken.

In a further aspect, a method of diagnosing or predicting the likelihood of occurrence of preterm birth (PTB) in a subject includes the steps of collecting a serum sample from a subject during a specific time of pregnancy and measuring differences in levels of markers of the panel compared to a control. The levels of one or more of the markers are measured by means of one or more of an ELISA, chromatography, mass spectrometry, and a cholesterol test. The specific time of sample collection may be in the first trimester, the second trimester (15-20 weeks), or the third trimester. In one embodiment, the specific time is the second trimester.

In one embodiment, a method of diagnosing or predicting the likelihood of occurrence of preterm birth (PTB) in a subject may include the steps of collecting a serum sample from a subject during the second trimester of pregnancy, measuring the levels of a first marker from a first group of markers and a second marker from a second group of markers, calculating the differences in levels of the first marker and the second marker compared to a control, and determining the likelihood of occurrence of preterm birth (PTB) in the subject. As discussed above, a significant decrease in the first marker and a significant increase in the second marker measured in serum taken from a subject during the second trimester of pregnancy compared to a control are diagnostic or predictive of an increased risk of preterm birth for the subject.

Additional markers may be included in the diagnostic methodologies contemplated herein to predict preterm birth. For example, pregnancy associated plasma protein A (PAPP-A), human chorionic gonadotropin (hCG) measured in the first trimester, and estriol, AFP, inhibin A and hCG measured in the second trimester may be measured. It is believed that reduced levels of these markers are indicative of increased risk of preterm birth.

Further, additional markers may be selected from a lipid panel including total cholesterol (TC), low-density lipoprotein (LDL), high-density lipoprotein (HDL) and triglycerides (TG). It is believed that reduced levels of these markers are also indicative of increased risk of preterm birth. Accordingly, a lipid test is also contemplated herein.

In some cases, additional diagnostic criteria may be considered such as the level or the occurrence of the markers with the presentation of maternal characteristics. “Maternal characteristics” as used herein, include, but are not limited to, cervical length, maternal microbiome (including microbiota, among other possible sites, from the vagina and gastrointestinal tract), maternal race, ethnicity, weight (both trimesters), age and gestational age at sampling (both trimesters), education, ethnicity, race, smoking status, height, previous live births, previous PTB, diabetes (pre-pregnancy and gestational), pre-pregnancy hypertension and sexually transmitted diseases. Additional information may be considered such as cerclage, tocolysis, labor onset, prelabor rupture of membranes (PROM), induction, fetal presentation, congenital anomalies, birth weight, gestational age, and plurality.

Specifically, the maternal characteristic may be selected from the group consisting of age, gestational age, weight (both trimesters), education, ethnicity, race, smoking status, height, previous live births, previous PTB, diabetes (pre-pregnancy and gestational), pre-pregnancy hypertension and sexually transmitted diseases.

In other cases, the maternal characteristic is the information about treatment during pregnancy and delivery outcomes, wherein the information is selected from the group consisting of cerclage, tocolysis, labor onset, prelabor rupture of membranes (PROM), induction, fetal presentation, congenital anomalies, birth weight, gestational age, and plurality.

While the present disclosure is directed toward the identification of subjects at significantly increased risk of experiencing PTB, it is contemplated that the present invention also identifies subjects that most likely will experience full term birth.

Unless defined otherwise in this specification, technical and scientific terms used herein have the same meaning as commonly understood by one of ordinary skill in the art to which this invention belongs and by reference to published texts.

It should be understood that while various embodiments in the specification are presented using “comprising” language, under various circumstances, a related embodiment may also be described using “consisting of” or “consisting essentially of” language. It is to be noted that the term “a” or “an,” refers to one or more, for example, “an immunoglobulin molecule,” is understood to represent one or more immunoglobulin molecules. As such, the terms “a” (or “an”), “one or more,” and “at least one” is used interchangeably herein.

The following examples set forth preferred markers and methods in accordance with the invention. It is to be understood, however, that these examples are provided by way of illustration and nothing herein should be taken as a limitation upon the overall scope of the invention.

### EXAMPLES

### Example 1

### Metabolic Profile of Preterm and Term Pregnancy

The purpose of this example was to characterize the metabolic profile of second trimester human serum/plasma associated with either a resultant full-term or preterm birth. Global biochemical profiles were determined in human serum and plasma samples, comparing serum collected from women in their second trimester of a pregnancy that resulted in subsequent full-term or preterm birth; plasma samples representing full-term birth were compared to full-term serum samples.

| Group | N | Description |
|---|---|---|
| 1 | 10 | Full-term birth, plasma (control) |
| 2 | 10 | Full-term birth, serum (control) |
| 3 | 10 | Preterm birth, serum (case) |

The samples were inventoried, and immediately stored at −80° C. At the time of analysis samples were extracted and prepared for analysis using a standard solvent extraction method for preparations of samples for gas chromatography (GS)/mass spectrometry (MS) and liquid chromatography (LC)/MS/MS platforms. The extracted samples were split into equal parts for analysis on the GC/MS and LC/MS/MS platforms. Also included were several technical replicate samples created from a homogeneous pool containing a small amount of all study samples (“Client Matrix”). General platform methods are described below.

Instrument variability was determined by calculating the median relative standard deviation (RSD) for the internal standards that were added to each sample prior to injection into the mass spectrometers. Overall process variability was determined by calculating the median RSD for all endogenous metabolites (i.e., non-instrument standards) present in 100% of the Client Matrix samples, which are technical replicates of pooled client samples. Values for instrument and process variability meet predetermined acceptance criteria as shown in the table below.

| QC Sample | Measurement | Median RSD |
|---|---|---|
| Internal Standards | Instrument Variability | 8% |
| Endogenous Biochemicals | Total Process Variability | 12% |

The present dataset comprises a total of 386 compounds of known identity (named biochemicals). Following log transformation and imputation with minimum observed values for each compound, Welch's two-sample t-test was used to identify biochemicals that differed significantly between experimental groups. A summary of the numbers of biochemicals that achieved statistical significance (p≦0.05), as well as those approaching significance (0.05<p<0.10), is shown below.

An estimate of the false discovery rate (q-value) is calculated to take into account the multiple comparisons that normally occur in metabolomic-based studies. For example, when analyzing 200 compounds, we would expect to see about 10 compounds meeting the p<0.05 cut-off by random chance. The q-value describes the false discovery rate; a low q-value (q<0.10) is an indication of high confidence in a result. While a higher q-value indicates diminished confidence, it does not necessarily rule out the significance of a result. Other lines of evidence may be taken into consideration when determining whether a result merits further scrutiny. Such evidence may include a) significance in another dimension of the study, b) inclusion in a common pathway with a highly significant compound, or c) residing in a similar functional biochemical family with other significant compounds.

Statistical Comparisons
Welch's Two-Sample t-Test

| Significantly | | |
|---|---|---|
| Altered | Preterm (serum) | Full-term (plasma) |
| Biochemicals | F̅u̅l̅l̅-̅t̅e̅r̅m̅ (̅s̅e̅r̅u̅m̅)̅ | Full-term (serum) |
| Total biochemicals | 66 | 268 |
| p ≦ 0.05 | | |
| Biochemicals | 22(↑)\|44(↓) | 60(↑)\|208(↓) |
| Total biochemicals | 28 | 20 |
| 0.05 < p < 0.10 | | |
| Biochemicals | 8(↑)\|20(↓) | 4(↑)\|16(↓) |

Despite the advanced state of medical care, approximately 1 in 8 babies born in the United States each year are preterm, or born prior to 37 weeks gestation. While some risk factors for preterm birth have been identified, most premature births have no known cause and involve women with no known risk factors. The ability to identify women at risk for preterm birth would allow for early interventions to reduce health risks and improve outcomes for mothers and babies. The purpose of this study was to profile the global serum metabolome of pregnant women sampled in the second trimester who went on to experience full-term (n=10) or preterm birth (n=10). In addition, 10 second trimester plasma samples from pregnant women who went on to experience full-term birth were also provided for comparison. Individual serum and plasma samples were loaded in equivalent volumes across the platform with no additional normalization performed prior to statistical analysis.

RF analysis of serum biochemical profiles differentiated the preterm and full-term (serum) groups with an overall predictive accuracy of 90%. RF is an unbiased and supervised classification technique based on an ensemble of a large number of decision trees. Using the primary groupings of preterm or full-term, RF classification analysis of serum metabolic profiles resulted in 90% accuracy in differentiating the two groups. The outcome of this RF analysis was better than random chance alone (50% accuracy), indicating that differences in serum biochemical profiles between the two groups were quite pronounced. RF analysis also produces a list of biochemicals ranked by their importance to the classification scheme. Among the 30 top-ranking metabolites resulting from the RF analysis, biochemicals related to vitamins (antioxidants), lipid metabolism, amino acid metabolism, energy metabolism, nucleotide metabolism, and carbohydrate metabolism were identified as contributing significantly to separation of the two groups. The details and implications of these metabolites identified in the RF analyses and other biochemical differences are discussed below.

Lipid-derived eicosanoids were elevated in the preterm birth group. Some of the largest and most consistent differences observed in the dataset included significant increases in several ω-6 fatty acid-derived inflammatory eicosanoids. For example, the linoleate (18:2n6)-derived markers of lipid peroxidation 13-HODE+9-HODE were higher in women who went on to experience preterm birth, as were several inflammatory eicosanoids derived from arachidonate (20:4n6) including prostaglandins A2 and E2, leukotriene B4, and 5-HETE. In addition, oxidation of 5-HETE results in production of a major marker of oxidative stress, 5-oxoETE, which was increased more than 6-fold in the preterm birth group. As prostaglandins play an important role in pregnancy-induced hypertension (preeclampsia/eclampsia) and are involved in preparing the cervix and uterus for labor and delivery, second trimester elevations in these prostaglandins may serve as biomarkers of impending preterm birth. Indeed, all of these lipid-derived eicosanoids were included in the RF importance plot as biochemicals that were important for separation of groups. In addition to elevations in markers of inflammation and oxidative stress, depletion of all forms of the powerful antioxidant vitamin E (α-tocopherol, β-tocopherol, and γ-tocopherol) were also observed in the preterm birth group and α-tocopherol was identified in the RF analysis as the biochemical that contributed most to distinguishing the two groups. Taken together, these findings indicate that pronounced differences in inflammation, oxidative stress, and antioxidant capacity are present at early time points in women who go on to experience preterm birth.

Bradykinin metabolism was altered in women who go on to experience preterm birth. Bradykinin is a potent vasodilatory peptide formed by the proteolytic cleavage of kininogen by kallikrein that may protect against ischemic injury. The enzyme kininase I can further transform bradykinin to an active metabolite, bradykinin, des-arg(9), which also reduces vascular resistance and acts as a hypotensive agent. Interestingly, the significant reduction in bradykinin and significant elevation in bradykinin, des-arg (9) in the preterm group is suggestive of altered bradykinin metabolism in these women. In addition, accumulation of the bradykinin-related polypeptides HWESASXX (SEQ ID NO:1), XHWESASXXR (SEQ ID NO:2), and HWESASLLR (SEQ ID NO:3) provides additional evidence of differences in blood flow and possibly coagulation in women who go on to experience preterm birth. It is important to note that the observed differences in prostaglandins may also contribute to altered vascular resistance and blood flow in the preterm group.

Altered cholesterol and steroid metabolism is observed in the preterm birth group. Pronounced hormonal changes occur in pregnancy, including alterations in the levels of many sex steroid hormones that are involved in various physiological processes. In the current study, a significant reduction in circulating cholesterol was observed in the preterm birth group with additional differences in cholesterol derivatives and sulfated steroid hormones. For example, the major bile acid precursor 7-alpha-hydroxycholesterol was significantly higher and a related metabolite involved in bile acid synthesis, 7-alpha-hydroxy-3-oxo-4-cholestenoate (7-HOCA), was lower in women who went on to experience preterm birth. These differences may be suggestive of altered hepatic bile acid synthesis that has not yet translated to differences in circulating bile acid levels. Accumulation of the pro-oxidative and cytotoxic metabolite 7-beta-hydroxycholesterol, which is present in oxidized low-density lipoprotein (LDL) particles, provides additional support for the notion of an inflammatory and oxidative environment in the preterm birth group. Finally, reductions in several sulfate-conjugated steroid hormones (androsterone sulfate, 4-androsten-3beta,17beta-diol disulfate 2, and andro steroid monosulfate 2) may be related to altered synthesis and/or changes in detoxification and clearance, which predominantly occurs in the liver. Of note, the alpha- and beta-hydroxylated cholesterol derivatives, dehydroisoandrosterone sulfate (DHEA-S), and cholesterol were all included in the importance plot generated from the RF analysis.

Changes in methionine metabolism and γ-glutamyl amino acids: implications for glutathione metabolism and liver function. The essential amino acid methionine serves as a precursor for cysteine, which is the rate-limiting biochemical for synthesis of the powerful antioxidant glutathione. Interestingly, in the preterm group significant reductions in circulating levels of both methionine and cysteine were observed, which has the potential to affect glutathione synthesis and therefore antioxidant capacity. Evidence for alterations in γ-glutamyl cycle activity, which functions predominantly in the liver, involves the enzyme γ-glutamyltransferase (GGT), and is responsible for amino acid transport across membranes, leukotriene metabolism, and glutathione recycling, was noted in the preterm group as well. Elevations in all γ-glutamyl amino acids may be suggestive of increased GGT activity, which is a common clinical finding in individuals with potential liver dysfunction. Increased GGT activity may also be indicative of greater glutathione turnover, possibly associated with elevated oxidative demands and/or reduced synthesis due to limited cysteine, in the preterm birth group. In addition to accumulation of γ-glutamyl amino acids, significant reductions in serum bilirubin (Z,Z) and bilirubin (E,E) provide further support for the notion of altered hepatic function in women who go on to experience preterm birth.

Altered branched-chain amino acid (BCAA) catabolism is observed in women who go on to experience preterm birth. The BCAAs isoleucine, leucine, and valine constitute a large portion of the amino acids stored in skeletal muscle and are an important source of energy in times of high demand. The BCAAs are first metabolized to their α-keto acid derivatives by cystosolic branched-chain aminotransferase (BCAT) and then subsequently degraded in mitochondria through the branched-chain α-keto acid dehydrogenase complex (BCKD) and other enzymes to acetyl-CoA or succinyl-CoA for eventual entry into the Krebs cycle. An interesting observation in the preterm group included significant reductions in the α-keto acids (3-methyl-2-oxobutyrate, 3-methyl-2-oxovalerate, and 4-methyl-2-oxopentanoate) despite the lack of change in the BCAAs or other metabolites related to BCAA catabolism. Although the meaning behind this difference is unclear, these findings suggest that early alterations in BCAT and/or BCKD enzyme activity may be present in women who go on to experience preterm birth. Importantly, all three of the α-keto acid derivates were included in the RF importance plot, suggesting that this unusual yet consistent difference is important for separation of the preterm and full-term groups.

Differences Observed when Comparing Plasma and Serum Samples from Women with Full-Term Pregnancies.

Overall, a greater number of metabolites were detected in serum as compared to plasma, with a loss of several dipeptides, bradykinin-related polypeptides, dicarboxylic fatty acids, eicosanoids, monoacylglycerols, and xenobiotics/drug-related metabolites from the plasma dataset. Conversely, several biochemicals in the carbohydrate superpathway were measured in plasma but not serum samples. In general, metabolite levels in plasma samples were lower than those in serum, with the notable exceptions of the fibrinogen cleavage peptides (related to clotting), many carbohydrates, citrate, and the long-chain acylcarnitines.

Application of PCA to determine separation of plasma and serum samples demonstrated that the two groups were clearly distinguishable. In this analysis, a large number of metabolic variables were transformed into a smaller number of orthogonal variables (i.e., Comp. 1, Comp. 2, Comp. 3) in order to analyze variation between the two groups. Taken together, the PCA and large number of differences between the two groups (69% of biochemicals changed significantly) demonstrate the importance of consistency in sample collection for metabolomics studies. Statistical comparisons between plasma samples from women who experienced full-term birth and serum from women who experienced preterm birth were not conducted due to the fact that the majority of changes would be attributable to differences in the matrices as opposed to true biochemical alterations associated with preterm birth.

In conclusion, significant and interesting metabolic differences were observed when comparing serum from women who went on to experience preterm or full-term births. RF analysis demonstrated impressive separation between the two groups, and many of the metabolites identified in the importance plot were consistent with metabolic pathways that were clearly altered in the preterm birth group. Changes in inflammatory and pro-oxidative eicosanoids and bradykinin metabolism may have important implications for blood pressure regulation and coagulation in pregnant women. Furthermore, alterations in antioxidant capacity and γ-glutamyl cycle activity may be evident as early as the second trimester in women who go on to experience preterm birth. It is important to note the reoccurring theme of possible hepatic dysfunction in the preterm birth group that emerged as evidenced by differences in bradykinin (precursor protein is synthesized in the liver) metabolism, bile acid synthesis, steroid hormone metabolism/sulfation, γ-glutamyl cycle activity, and serum bilirubin. Validation of these exciting findings in an independent cohort of women sampled during their second trimester has the potential to confirm early serum biomarkers of preterm birth and address this significant unmet medical need that affects millions of women and babies worldwide.

Sample Accessioning:

Each sample received was accessioned into the Metabolon LIMS system and was assigned by the LIMS a unique identifier, which was associated with the original source identifier only. This identifier was used to track all sample handling, tasks, results etc. The samples (and all derived aliquots) were bar-coded and tracked by the LIMS system. All portions of any sample were automatically assigned their own unique identifiers by the LIMS when a new task was created; the relationship of these samples was also tracked. All samples were maintained at −80° C. until processed.

Sample Preparation:

The sample preparation process was carried out using the automated MicroLab STAR® system from Hamilton Company. Recovery standards were added prior to the first step in the extraction process for QC purposes. Sample preparation was conducted using a proprietary series of organic and aqueous extractions to remove the protein fraction while allowing maximum recovery of small molecules. The resulting extract was divided into two fractions; one for analysis by LC and one for analysis by GC. Samples were placed briefly on a TurboVap® (Zymark) to remove the organic solvent. Each sample was then frozen and dried under vacuum. Samples were then prepared for the appropriate instrument, either LC/MS or GC/MS.

QA/QC:

For QA/QC purposes, a number of additional samples are included with each day's analysis. Furthermore, a selection of QC compounds is added to every sample, including those under test. These compounds are carefully chosen so as not to interfere with the measurement of the endogenous compounds. Tables 1 and 2 describe the QC samples and compounds. These QC samples are primarily used to evaluate the process control for each study as well as aiding in the data curation.

TABLE 1
Description of QC Samples

| Type | Description | Purpose |
|---|---|---|
| MTRX | Large pool of human | Assure that all aspects of |
| | plasma maintained by | Metabolon process are operating |
| | Metabolon that has | within specifications. |
| | been characterized | |
| | extensively. | |
| CMTRX | Pool created by tak- | Assess the effect of a non-plasma |
| | ing a small aliquot | matrix on the Metabolon process |
| | from every customer | and distinguish biological vari- |
| | sample. | ability from process variability. |
| PRCS | Aliquot of ultra-pure | Process Blank used to assess the |
| | water | contribution to compound signals |
| | | from the process. |
| SOLV | Aliquot of solvents | Solvent blank used to segregate |
| | used in extraction. | contamination sources in the ex- |
| | | traction. |

TABLE 2
QC Standards

| Type | Description | Purpose |
|---|---|---|
| DS | Derivatization | Assess variability of derivatization for |
| | Standard | GC/MS samples. |
| IS | Internal | Assess variability and performance of |
| | Standard | instrument. |
| RS | Recovery | Assess variability and verify performance |
| | Standard | of extraction and instrumentation. |

Liquid Chromatography/Mass Spectrometry (LC/MS, LC/MS²):

The LC/MS portion of the platform was based on a Waters ACQUITY UPLC and a Thermo-Finnigan LTQ mass spectrometer, which consisted of an electrospray ionization (ESI) source and linear ion-trap (LIT) mass analyzer. The sample extract was split into two aliquots, dried, then reconstituted in acidic or basic LC-compatible solvents, each of which contained 11 or more injection standards at fixed concentrations. One aliquot was analyzed using acidic positive ion optimized conditions and the other using basic negative ion optimized conditions in two independent injections using separate dedicated columns. Extracts reconstituted in acidic conditions were gradient eluted using water and methanol both containing 0.1% Formic acid, while the basic extracts, which also used water/methanol, contained 6.5 mM ammonium bicarbonate. The MS analysis alternated between MS and data-dependent MS² scans using dynamic exclusion.

Gas chromatography/Mass Spectrometry (GC/MS):

The samples destined for GC/MS analysis were re-dried under vacuum desiccation for a minimum of 24 hours prior to being derivatized under dried nitrogen using bistrimethyl-silyl-triflouroacetamide (BSTFA). The GC column was 5% phenyl and the temperature ramp is from 40° to 300° C. in a 16 minute period. Samples were analyzed on a Thermo-Finnigan Trace DSQ fast-scanning single-quadrupole mass spectrometer using electron impact ionization. The instrument was tuned and calibrated for mass resolution and mass accuracy on a daily basis. The information output from the raw data files was automatically extracted as discussed below.

Accurate Mass Determination and MS/MS Fragmentation (LC/MS), (LC/MS/MS):

The LC/MS portion of the platform was based on a Waters ACQUITY UPLC and a Thermo-Finnigan LTQ-FT mass spectrometer, which had a linear ion-trap (LIT) front end and a Fourier transform ion cyclotron resonance (FT-ICR) mass spectrometer backend. For ions with counts greater than 2 million, an accurate mass measurement could be performed. Accurate mass measurements could be made on the parent ion as well as fragments. The typical mass error was less than 5 ppm. Ions with less than two million counts require a greater amount of effort to characterize. Fragmentation spectra (MS/MS) were typically generated in data dependent manner, but if necessary, targeted MS/MS could be employed, such as in the case of lower level signals.

Bioinformatics:

The informatics system consisted of four major components, the Laboratory Information Management System (LIMS), the data extraction and peak-identification software, data processing tools for QC and compound identification, and a collection of information interpretation and visualization tools for use by data analysts. The hardware and software foundations for these informatics components were the LAN backbone, and a database server running Oracle 10.2.0.1 Enterprise Edition.

Data Extraction and Quality Assurance:

The data extraction of the raw mass spec data files yielded information that could loaded into a relational database and manipulated without resorting to BLOB manipulation. Once in the database the information was examined and appropriate QC limits were imposed. Peaks were identified using proprietary peak integration software, and component parts were stored in a separate and specifically designed complex data structure.

Compound Identification:

Compounds were identified by comparison to library entries of purified standards or recurrent unknown entities. Identification of known chemical entities was based on comparison to metabolomic library entries of purified standards. The combination of chromatographic properties and mass spectra gave an indication of a match to the specific compound or an isobaric entity. Additional entities could be identified by virtue of their recurrent nature (both chromatographic and mass spectral). These compounds have the potential to be identified by future acquisition of a matching purified standard or by classical structural analysis.

Curation:

A variety of curation procedures were carried out to ensure that a high quality data set was made available for statistical analysis and data interpretation. The QC and curation processes were designed to ensure accurate and consistent identification of true chemical entities, and to remove those representing system artifacts, mis-assignments, and background noise.

Proprietary visualization and interpretation software were used to confirm the consistency of peak identification among the various samples. Library matches for each compound were checked for each sample and corrected if necessary.

Normalization:

For studies spanning multiple days, a data normalization step was performed to correct variation resulting from instrument inter-day tuning differences. Essentially, each compound was corrected in run-day blocks by registering the medians to equal one (1.00) and normalizing each data point proportionately (termed the “block correction”). For studies that did not require more than one day of analysis, no normalization is necessary, other than for purposes of data visualization.

Statistical Calculation:

For many studies, two types of statistical analysis are usually performed: (1) significance tests and (2) classification analysis. (1) For pair-wise comparisons we typically perform Welch's t-tests and/or Wilcoxon's rank sum tests. For other statistical designs we may perform various ANOVA procedures (e.g., repeated measures ANOVA). (2) For classification we mainly use random forest analyses. Random forests give an estimate of how well we can classify individuals in a new data set into each group, in contrast to a t-test, which tests whether the unknown means for two populations are different or not. Random forests create a set of classification trees based on continual sampling of the experimental units and compounds. Then each observation is classified based on the majority votes from all the classification trees. Statistical analyses are performed with the program “R” http://cran.r-project.org/.

### Example 2

### Metabolic Markers in Human Maternal Serum for Predicting Preterm Birth

A dataset comprising a total of 343 compounds of known identity (named biochemicals) were tested by the procedure described in Example 1. The purpose of this example was to profile the global serum metabolome of pregnant women sampled in the second trimester who went on to experience term (n=40) or preterm (n=40) birth. Individual serum samples were loaded in equivalent volumes across the platform with no additional normalization performed prior to statistical analysis.

| Group | n | Description |
|---|---|---|
| Preterm | 40 | Preterm labor (<35 week pregnancy) |
| Term | 40 | Term labor (40 week pregnancy) |

Instrument variability was determined by calculating the median relative standard deviation (RSD) for the internal standards that were added to each sample prior to injection into the mass spectrometers. Overall process variability was determined by calculating the median RSD for all endogenous metabolites (i.e., non-instrument standards) present in 100% of the Client Matrix samples, which are technical replicates of pooled client samples. Values for instrument and process variability are shown in the table below.

| QC Sample | Measurement | Median RSD |
|---|---|---|
| Internal Standards | Instrument Variability | 5% |
| Endogenous Biochemicals | Total Process Variability | 11% |

Following log transformation and imputation with minimum observed values for each compound, Welch's two-sample t-test was used to identify biochemicals that differed significantly between experimental groups. A summary of the numbers of biochemicals that achieved statistical significance (p≦0.05), as well as those approaching significance (0.05<p<0.10), is shown below.

An estimate of the false discovery rate (q-value) is calculated to take into account the multiple comparisons that normally occur in metabolomic-based studies. For example, when analyzing 200 compounds, it was expected to see about 10 compounds meeting the p≦0.05 cut-off by random chance. The q-value describes the false discovery rate; a low q-value (q<0.10) is an indication of high confidence in a result. While a higher q-value indicates diminished confidence, it does not necessarily rule out the significance of a result.

Statistical Comparisons
Welch's Two-Sample t-Test

| Significantly Altered | Preterm |
|---|---|
| Biochemicals | Term |
| Total biochemicals | 103 |
| p ≦ 0.05 | |
| Biochemicals | 43\|60 |
| (↑↓) | |
| Total biochemicals | 14 |
| 0.05 < p < 0.10 | |
| Biochemicals | 2\|12 |
| (↑↓) | |

This example first presents statistical classification of the term and preterm groups by principal component analysis (PCA) and random forest analysis, followed by a discussion of metabolic pathways and biochemicals that differed between the two groups, as described below.

PCA revealed clear separation between the term and preterm groups. Application of PCA to determine separation of second trimester serum samples from women who went on to experience term or preterm birth demonstrated that the two groups were readily distinguishable. In this analysis, a large number of metabolic variables were transformed into a smaller number of orthogonal variables (i.e., Comp. 1, Comp. 2) in order to analyze variation between the two groups and populations that differ are expected to cluster separately. Findings from this PCA corroborate the clear separation of term and preterm groups observed in the PCA conducted for the pilot study.

RF analysis of serum biochemical profiles differentiated the term and preterm groups with an overall predictive accuracy of 89%. RF is an unbiased and supervised classification technique based on an ensemble of a large number of decision trees. Using the primary groupings of term or preterm, RF classification analysis of serum metabolic profiles resulted in 89% accuracy in differentiating the two groups. The outcome of this RF analysis was better than random chance alone (50% accuracy), indicating that differences in serum biochemical profiles between the two groups were quite pronounced. RF analysis also produces a list of biochemicals ranked by their importance to the classification scheme. Among the 30 top-ranking metabolites resulting from the RF analysis, biochemicals related to amino acid/peptide metabolism, lipid metabolism, and cofactors/vitamins were identified as contributing significantly to separation of the two groups. Importantly, 12 metabolites included in the current RF importance plot were also identified as contributing to the separation of groups in the RF analysis run for the pilot study.

Eicosanoids and markers of lipid peroxidation/inflammation, including 13-HODE +9-HODE, leukotriene B4, prostaglandin E2, 5-HETE, and 5-oxoETE were significantly elevated in the preterm group. Additional related metabolites such as the linoleic acid-/lipoxygenase-derived oxylipin 9(S)-hydroperoxy-10E,12Z-octadecadienoic acid [9(S)-HpODE], eicosanoid 5-HEPE, and oxidative stress marker methionine sulfoxide were identified in this follow-up study and were also significantly higher in the preterm group. Also circulating levels of the vitamin E isoforms alpha-tocopherol, beta-tocopherol, and gamma-tocopherol were significantly reduced in the preterm group. An additional vitamin E metabolite, gamma-CEHC, was also significantly lower in the preterm group. Taken together, these findings are suggestive of either reduced antioxidant capacity or depleted antioxidant reserves in these women.

Accumulation of bradykinin-related peptides such as HWESASXX (SEQ ID NO:1) and HXGXA (SEQ ID NO:4) were noted in the preterm group. In addition, altered levels of cholesterol-derived biochemicals, including the pro-oxidative and cytotoxic metabolite 7-beta-hydroxycholesterol and bile acid precursor 7-alpha-hydroxy-3-oxo-4-cholestenoate (7-Hoca), were again observed in the preterm group. In the current study, the sulfated secondary bile acids glycolithocholate sulfate and taurolithocholate 3-sulfate were significantly lower in the preterm group, but these differences may also be related to alterations in gut microbial metabolism (as discussed later in the report) and/or changes in sulfation pathways, and therefore liver function, in women who go on to experience preterm birth. With regard to potential changes in liver function, consistent elevations in many gamma-glutamyl amino acids, which may be indicative of increased hepatic γ-glutamyltransferase (GGT) activity, and significantly lower levels of bilirubin (E,E) and biliverdin were observed in both the pilot study and the current follow-up study.

Branched-chain amino acid (BCAA) catabolism was altered in the preterm group. Specifically, degradation of the BCAAs valine, isoleucine, and leucine was altered in the current study. In addition to depletion of the three α-keto acids 3-methyl-2-oxobutyrate, 3-methyl-2-oxovalerate, and 4-methyl-2-oxopentanoate, levels of several related metabolites were significantly lower in the preterm group. For example, the valine-derived metabolites isobutyrylcarnitine and 3-hydroxyisobutyrate, the isoleucine-derived metabolite 2-methylbutyrylcarnitine, and the leucine-derived metabolite isovalerylcarnitine were reduced in the preterm group. In addition, the BCAA- and fatty acid-derived carnitine conjugate of propionyl-CoA, propionylcarnitine, was also significantly lower in the preterm group. As BCAAs constitute up to 35% of muscle content, and BCAAs serve as an important energy source during times of need, these differences may be suggestive of early alterations in muscle/energy metabolism in women who go on to experience preterm birth. Alternatively, BCAAs are primarily degraded in the mitochondria and general reductions in BCAA-related metabolites may be reflective of mitochondrial dysfunction in the preterm group, although the specific tissue(s) exhibiting this phenomenon cannot be identified since serum is a “sink” for all metabolic processes occurring in the body. Finally, BCAAs are thought to play a role in insulin secretion from pancreatic β-cells and altered BCAA metabolism may contribute to changes in maternal insulin sensitivity. This connection may be of significance as women diagnosed with gestational diabetes are at an increased risk for preterm birth.

Changes in serum levels of metabolites derived from gut flora in the preterm group. The gut microbiota is increasingly being recognized for its role in health and disease. The role of gut flora in pregnancy is especially pronounced, as the composition changes each trimester and is transferred from mother to baby (when born vaginally) in the birth canal, which contributes to development of a healthy immune system. In the current study, significant reductions in circulating levels of several amino acid metabolites produced by activity of the gut microbiome were observed in the preterm group. For example, levels of the phenylalanine degradation products phenyllactate (PLA) and phenylacetylglutamine, tyrosine degradation product p-cresol sulfate, and tryptophan degradation products indolepropionate (IPA) and 3-indoxyl sulfate were lower in the preterm group. Taken together, these findings are indicative of early alterations in composition and/or activity of gut flora in women who go on to experience preterm birth However, it is important to note that levels of the sulfated metabolites p-cresol sulfate and 3-indoxyl sulfate may also be reflective of changes in hepatic function. Furthermore, the observed differences in serum levels of gut bacterial-derived metabolites may be associated with changes in gut wall permeability, which can play an important role in inflammation and disease.

Potential alterations in protein metabolism in the preterm group. Serum levels of many metabolites reflective of protein metabolism were different when comparing the term and preterm groups. Furthermore, the majority of biochemicals identified in the RF importance plot as contributing to the separation of groups were related to amino acid/peptide metabolism. For example, a number of dipeptides, which may be indicative of protein degradation, were elevated in the preterm group. This was accompanied by an elevation in the amino acid arginine and a reduction in serum urea, metabolites that are critical for operation of the urea cycle. The urea cycle occurs predominantly in liver and kidney and is important for detoxification of toxic nitrogenous waste (ammonia) produced from deamination of amino acids and nucleic acids, which is converted to urea for subsequent excretion in urine. Although a universal elevation in free amino acids was not present, elevations in dipeptides and lower urea levels may be indicative of altered urea cycle activity, and therefore changes in liver and/or kidney function in women who go on to experience preterm birth. In addition, the observed difference in serum urea levels may be related to altered BCAA catabolism as well.

In conclusion, the larger sample size also resulted in identification of a greater number of significant biochemical differences (Δ=103) as compared to the pilot study (Δ=66) when contrasting the term and preterm groups. For example, serum levels of cotinine, a nicotine metabolite that serves as a biomarker for tobacco smoke exposure, was non-significantly elevated in the preterm group in the pilot study but was significantly higher in the preterm group in the current follow-up study. Furthermore, blinded classification of the 80 follow-up samples based on the original RF analysis conducted using the 20 pilot study samples gave excellent sensitivity (90%) with 36/40 correctly classified as “preterm” but lower specificity (68%) with 27/40 correctly classified as “term” (data sent early). Together with the impressive ability to distinguish the term and preterm groups by PCA and RF analysis in the current study, these multiple studies and analyses reveal that serum metabolic profiles measured in the second trimester are powerful tools for predicting eventual preterm birth.

### Example 3

### Identification of Metabolic Markers in Human Maternal Serum for Predicting Preterm Birth

I. Experimental Design

Global biochemical profiles were determined in human maternal serum samples, compared across gestational age cohorts as presented below.

| Group | n | Description |
|---|---|---|
| 24-31 | 34 | Gestational age = 24-31 weeks |
| 32-33 | 34 | Gestational age = 32-33 weeks |
| 34 | 34 | Gestational age = 34 weeks |
| 35 | 34 | Gestational age = 35 weeks |
| 36 | 34 | Gestational age = 36 weeks |
| 37 | 34 | Gestational age = 37 weeks |
| 38 | 34 | Gestational age = 38 weeks |
| 39 | 34 | Gestational age = 39 weeks |
| 40 | 34 | Gestational age = 40 weeks |
| 41 | 34 | Gestational age = 41 weeks |
| Blinded | 170 | Blinded samples, 17 from each gestational age group |

II. Summary of Procedure

A set of collected human serum samples were inventoried, and immediately stored at −80° C. At the time of analysis samples were extracted and prepared for analysis using a standard solvent extraction method. The extracted samples were split into equal parts for analysis on the GC/MS and LC/MS/MS platforms.

IV. Data Quality: Instrument and Process Variability

| QC Sample | Measurement | Median RSD |
|---|---|---|
| Internal Standards | Instrument Variability | 5% |
| Endogenous Biochemicals | Total Process Variability | 11% |

Instrument variability was determined by calculating the median relative standard deviation (RSD) for the internal standards that were added to each sample prior to injection into the mass spectrometers. Overall process variability was determined by calculating the median RSD for all endogenous metabolites (i.e., non-instrument standards) present in 100% of the Client Matrix samples, which are technical replicates of pooled client samples. Values for instrument and process variability meet acceptance criteria as shown in the table above.

IV. Metabolite Summary and Significantly Altered Biochemicals

The present dataset comprises a total of 479 compounds of known identity (named biochemicals). Following log transformation and imputation with minimum observed values for each compound, Welch's two-sample t-test was used to identify biochemicals that differed significantly between experimental groups. A summary of the numbers of biochemicals that achieved statistical significance (p≦0.05), as well as those approaching significance (0.05<p<0.10), is shown below.

An estimate of the false discovery rate (q-value) is calculated to take into account the multiple comparisons that normally occur in metabolomic-based studies. For example, when analyzing 200 compounds, we would expect to see about 10 compounds meeting the p≦0.05 cut-off by random chance. The q-value describes the false discovery rate; a low q-value (q<0.10) is an indication of high confidence in a result. While a higher q-value indicates diminished confidence, it does not necessarily rule out the significance of a result. Other lines of evidence may be taken into consideration when determining whether a result merits further scrutiny. Such evidence may include a) significance in another dimension of the study, b) inclusion in a common pathway with a highly significant compound, or c) residing in a similar functional biochemical family with other significant compounds.

Statistical Comparisons
Welch's Two-Sample t-Test

| Significantly Altered | 24-31 wks | 32-33 wks | 34 wks | 35 wks | 36 wks | 37 wks |
|---|---|---|---|---|---|---|
| Biochemicals | 38-41 wks | 38-41 wks | 3̅8̅-̅4̅1̅ w̅k̅s̅ | 3̅8̅-̅4̅1̅ w̅k̅s̅ | 3̅8̅-̅4̅1̅ w̅k̅s̅ | 3̅8̅-̅4̅1̅ w̅k̅s̅ |
| Total biochemicals | 133 | 141 | 154 | 128 | 141 | 23 |
| p ≦ 0.05 | | | | | | |
| Biochemicals | 83\|50 | 62\|79 | 73\|81 | 68\|60 | 62\|79 | 13\|10 |
| (↑↓) | | | | | | |
| Total biochemicals | 36 | 26 | 29 | 27 | 31 | 29 |
| 0.05 < p < 0.10 | | | | | | |
| Biochemicals | 18\|18 | 8\|18 | 7\|22 | 12\|15 | 7\|24 | 21\|8 |
| (↑↓) | | | | | | |

V. Biochemical Summary

The purpose of this study was to profile the global serum metabolome of pregnant women sampled in the second trimester who went on to experience pre-term or term birth across a continuum of gestational ages (GA) ranging from 24-41 weeks. Thirty-four serum samples were provided for each of the following ten groups defined by GA: 24-31, 32-33, 34, 35, 36, 37, 38, 39, 40, and 41 weeks, as well as 170 blinded samples (17 from each of the ten groups). Individual serum samples were loaded in equivalent volumes across the platform with no additional normalization performed prior to statistical analysis. It is important to note that two samples, KB201016119_{—}3_c_{—}1 (60 μl) and KB201210002_{—}3_c_{—}1 (80 μl) had limiting volumes and were therefore brought to 100 μl with water prior to sample processing. Identification of two linear regression models. Statistical modeling was carried out using GA as the outcome. Two least-squares regression models that included two explanatory variables (glutamate and methionine) and seven explanatory variables (beta-hydroxyisovalerate, glutamate, glycerol 2-phosphate, HWESASXX (SEQ ID NO:1), methionine, paraxanthine, and proline) were identified as having the highest correlations between actual and predicted GA. Additional details for the linear regression models and predictions of GA for blinded samples based on the linear regression analyses are provided as additional word (“Model Summaries.docx”) and excel (“All Predictions.xlsx”) files, respectively.

PCA Revealed a General Separation of Two GA Groupings: 24-36 Weeks and 37-41 Weeks.

Application of PCA (described herein elsewhere) to determine separation of serum samples from women who went on to experience birth across a range of GA demonstrated the emergence of two distinct groups: one group of women who delivered between 24-36 weeks and one group of women who delivered between 37-41 weeks. In this analysis, a large number of metabolic variables were transformed into a smaller number of orthogonal variables (i.e., Comp. 1, Comp. 2) in order to analyze variation between groups and populations that differ are expected to cluster separately. Findings from this PCA corroborate many of the metabolic patterns of difference observed in the current study, as well as the clear separation of term and preterm groups observed in PCA conducted for the previous studies.

RF Analysis of Serum Biochemical Profiles Differentiated Four GA Groups (24-33 Weeks, 34-36 Weeks, 37-38 Weeks, and 39-41 Weeks) with a Predictive Accuracy of 51% and Two GA Groups (24-36 Weeks and 37-41 Weeks) with a Predictive Accuracy of 87%.

RF is an unbiased and supervised classification technique based on an ensemble of a large number of decision trees. Using the four primary groupings of 24-33 weeks, 34-36 weeks, 37-38 weeks, and 39-41 weeks, RF classification analysis of serum metabolic profiles resulted in 51% accuracy in differentiating the four groups. The outcome of this RF analysis was better than random chance alone (25% accuracy), indicating that differences in serum biochemical profiles between the two groups were present. A second RF analysis comparing two GA groups, 24-36 weeks and 37-41 weeks, resulted in 87% accuracy in differentiating the two groups, which is also better than random chance alone (50% accuracy). RF analysis also produced a list of biochemicals ranked by their importance to the classification scheme. Among the 30 top-ranking metabolites resulting from the RF analyses, biochemicals related to amino acid/peptide metabolism, carbohydrate metabolism, lipid metabolism, cofactors/vitamins, energy metabolism, and xenobiotics were identified as contributing significantly to separation of groups. Importantly, several identical metabolites were included near the top of both RF importance plots (methionine, γ-glutamyl amino acids, and branched-chain α-keto acids) generated in the current study and were also identified as contributing to the separation of groups in RF analyses run for the previous studies.

Confirmation of Previous Findings from the Original Studies.

Many biochemical perturbations observed in the previous studies were confirmed in the current study, including identification of metabolites that previously demonstrated significant differences in the linear regression models and inclusion of common metabolites in the RF importance plots. For example, several eicosanoids and markers of lipid peroxidation/inflammation, including 13-HODE+9-HODE, leukotriene B4, prostaglandin E2, 5-HEPE, 5-HETE, 9-HETE, and 5-oxoETE were measured in the previous and current studies and were significantly elevated in all GA groups 36 weeks or less as compared to the 38-41 week group. Also in agreement with the previous studies, significant reductions in circulating levels of the vitamin E isoforms alpha-tocopherol, beta-tocopherol, and gamma-tocopherol and metabolite gamma-CEHC were observed in all GA groups 36 weeks or less as compared to the 38-41 week group. In this validation study, an additional biochemical marker of oxidative stress, allantoin, was significantly higher in the 24-31 week and 34 week groups as compared to the 38-41 week group. Elevations in allantoin, the final degradation product of purine nucleotide catabolism, were observed in the previous studies but these differences were not statistically significant. As humans lack the enzyme urate oxidase (UO) to convert urate to allantoin, this metabolite must be generated through non-enzymatic means involving reactive oxygen species and therefore acts as a biochemical marker of oxidative stress. Taken together, these findings support the previous observations of a pro-oxidative and pro-inflammatory state in women who go on to experience preterm birth and are suggestive of reduced antioxidant capacity in these women.

Also related to oxidative stress and altered redox homeostasis, differences in circulating levels of the glutathione precursors methionine and cysteine and the glutathione degradation product 5-oxoproline may be suggestive of altered glutathione metabolism in women who go on to experience preterm birth. Importantly, methionine was included in both linear regression models, was identified as the biochemical that contributed the most to separation of groups in both of the current RF analyses, and was included in RF importance plots generated from previous studies. In addition, consistent elevations in several γ-glutamyl amino acids (with the exception of γ-glutamylmethionine), which may be indicative of increased hepatic γ-glutamyltransferase (GGT) activity related to glutathione turnover, were noted in previous studies and in the current study. Elevated levels of γ-glutamyl amino acids and significantly lower levels of heme, bilirubin (Z,Z), bilirubin (E,E) and biliverdin, which were observed in the previous studies and in all GA groups 36 weeks or less as compared to the 38-41 week group, may also be indicative of a metabolic signature of altered hepatic function.

As was observed in the first pilot study, significant reductions in the potent vasodilatory peptide bradykinin in all GA groups 36 weeks or less as compared to the 38-41 week group and trending elevations in its active metabolite bradykinin, des-arg (9) in the 34 week, 35 week, and 36 week groups are suggestive of altered bradykinin metabolism in women who go on to experience preterm birth. In addition, accumulation of bradykinin-related polypeptides [HXGXA (SEQ ID NO:4), HWESASXX (SEQ ID NO:1), and HWESASLLR(SEQ ID NO:3)] provides additional evidence of differences in blood flow and possibly coagulation in these women.

Altered levels of cholesterol and several cholesterol-derived metabolites, including the pro-oxidative and cytotoxic metabolite 7-beta-hydroxycholesterol and bile acid precursors 7-alpha-hydroxycholesterol and 7-alpha-hydroxy-3-oxo-4-cholestenoate (7-Hoca), were again observed in all GA groups 36 weeks or less as compared to the 38-41 week group. Of note, in the current study many cholesterol-derived steroid hormones and sulfated metabolites were also altered in women who go on to experience preterm birth, although many of these differences were not consistent in all GA groups ranging from 24-36 weeks.

Alterations in amino acid metabolism and gut microbial metabolism were observed in all GA groups 36 weeks or less as compared to the 38-41 week group, which was also consistent across studies. For example, lower levels of free amino acids and related gut microbiome-derived metabolites, together with general elevations in circulating dipeptides, provide support for the notion of altered amino acid/protein metabolism. In addition, pronounced reductions in α-keto acids derived from the branched-chain amino acids (BCAAs), the BCAAs themselves, and other metabolites associated with BCAA catabolism were again observed in all GA groups 36 weeks or less as compared to the 38-41 week group.

Changes in Biochemicals Related to Energy Metabolism.

One interesting set of metabolic differences that emerged from this follow-up study included alterations in serum levels of a number of biochemicals reflective of energy metabolism. As serum acts a “sink” for all metabolic processes occurring in the body, directionality and tissue-specific changes cannot be determined from these findings. However, alterations in several metabolites related to glucose metabolism (the glycolytic intermediates glucose, pyruvate, and lactate and the pentitols ribose and arabinose that are synthesized through the pentose phosphate pathway), β-oxidation of fatty acids (medium-chain fatty acids that do not require carnitine conjugation for entry into the mitochondria and metabolites produced from degradation of fatty acids and BCAAs including propionylcarnitine, butyrylcarnitine, and isovalerate), amino acid anaplerosis, and TCA cycle activity (alpha-ketoglutarate and succinate) were observed in GA groups 36 weeks or less as compared to the 38-41 week group. Consistent with the general reductions in metabolites reflective of energy metabolism in women who go on to experience preterm birth, significantly lower levels of acetylcarnitine, the carnitine-conjugated derivative of the key energy molecule acetyl-CoA, were also observed. While clear alterations in energy metabolism are apparent, determination of the source and directionality of these changes in serum warrants additional investigation.

Changes in Tryptophan Metabolism.

The amino acid tryptophan serves as an important precursor for synthesis of bioactive metabolites, including the neurotransmitter serotonin (5HT). Interestingly, significant reductions in both tryptophan and serotonin were observed in all GA groups 36 weeks or less as compared to the 38-41 week group. Insufficient tryptophan and serotonin levels have been linked with depression in pregnancy (which is associated with preterm birth) and serotonin also plays a role in prenatal central nervous system development. Significant reductions in kynurenine, another tryptophan metabolite that is typically produced in the liver via the enzyme tryptophan dioxygenase (TDO) but that can also be synthesized through extrahepatic expression of a similar enzyme, indoleamine 2,3-dioxygenase (IDO) that is highly inducible by pro-inflammatory cytokines such as interferon-γ (IFNγ) and tumor necrosis factor-α (TNF-α), are not in agreement with the strong inflammatory signature noted in women who go on to experience pre-term birth. One potential explanation for this discrepancy may be related to simple depletion of the necessary precursor tryptophan. Alternatively, elevations in anthranilate, which is produced from kynurenine via the enzyme kynureninase, suggest that accelerated conversion of kynurenine may be responsible for the lower levels that were observed. Anthranilate is an intermediate for synthesis of the ubiquitous energy cofactor NAD+ from tryptophan and accumulation of this metabolite may potentially be related to changes in energy metabolism as well.

In conclusion, results from this well-powered, follow-up metabolic profiling study demonstrate strong confirmatory findings that support the previous studies. In addition, notable changes in metabolites related to energy metabolism and tryptophan degradation were observed in the current study. Based on the global serum metabolome, clear separation of samples obtained from women who went on to experience preterm birth (weeks 24-36) was observed when comparing to samples obtained from women who gave birth to full-term infants (weeks 37-41). Both statistical classification of the data and comparisons of individual metabolites among different GA groups confirmed this stark division between the 24-36 week and 37-41 week groups. In addition, combining of all datasets to date constitutes an extremely powerful approach for clearly defining metabolic perturbations and identifying serum biomarkers indicative of preterm birth. Moreover, these data provide a robust data set for assembly of diagnostic panels for screening women for risk of preterm birth.

### REFERENCES

1. Liu L, Johnson H L, Cousens S, et al. Global, regional, and national causes of child mortality: an updated systematic analysis for 2010 with time trends since 2000. The Lancet. 2012; 6736(12):1-11.
2. Goldenberg R L, Culhane J F, Iams J D, Romero R. Epidemiology and causes of preterm birth. The Lancet. 2008; 371(9606):75-84.
3. Berghella V. Universal Cervical Length Screening for Prediction and Prevention of Preterm Birth. Obstetrical & Gynecological Survey. 2012; 67(10):653-657.
4. Cahill A G, Odibo A O, Caughey A B, et al. Universal cervical length screening and treatment with vaginal progesterone to prevent preterm birth: a decision and economic analysis. American Journal of Obstetrics & Gynecology. 2010; 202(6):548.e1-8.
5. Iams J D, Berghella V. Care for women with prior preterm birth. American Journal of Obstetrics and Gynecology. 2010; 203(2):89-100.
6. Romero R, Nicolaides K, Conde-Agudelo A, et al. Vaginal progesterone in women with an asymptomatic sonographic short cervix in the midtrimester decreases preterm delivery and neonatal morbidity: a systematic review and metaanalysis of individual patient data. American Journal of Obstetrics and Gynecology. 2012; 206(2): 124. e1-19.
7. Honest H, Forbes C A, Durée K H, et al. Screening to prevent spontaneous preterm birth: systematic reviews of accuracy and effectiveness literature with economic modelling. Health Technology Assessment. 2009; 13(43):1-627.
8. Edison R J, Berg K, Remaley A, et al. Adverse birth outcome among mothers with low serum cholesterol. Pediatrics. 2007; 120(4):723-33.
9. Catov J M, Ness R B, Wellons M F, Jacobs D R, Roberts J M, Gunderson E P. Prepregnancy lipids related to preterm birth risk: the coronary artery risk development in young adults study. Clinical Endocrinology. 2010; 95(8):3711-8.
10. Tuckey R C. Progesterone synthesis by the human placenta. Placenta. 2005; 26(4):273-81.
11. Dugoff L, Hobbins J C, Malone F D, et al. Quad Screen as a Marker of Adverse Pregnancy Outcome. Obstetrics & Gynecology. 2005; 106(2):260-267.
12. Yuan W, Chen L, Bernal A L. Is elevated maternal serum alpha-fetoprotein in the second trimester of pregnancy associated with increased preterm birth risk? A systematic review and meta-analysis. European Journal of Obstetrics, Gynecology, and Reproductive Biology. 2009; 145(1):57-64.

We claim:

1. A panel for diagnosing or predicting the likelihood of occurrence of preterm birth (PTB) in a subject, the panel comprising at least one of:
(a) a first marker from a first group of markers that when significantly lower in concentration compared to a term birth control is predictive of preterm birth; and
(b) a second marker from a second group of markers that when significantly higher in concentration compared to the term birth control is predictive of preterm birth,
wherein a significant decrease in the first marker and a significant increase in the second marker measured in serum taken from a subject during the second trimester of pregnancy compared to a control is diagnostic or predictive of an increased risk of preterm birth for the subject.

2. The panel of claim 1, wherein the panel includes a marker from the first group and a marker from the second group.

3. The panel of claim 1, wherein the panel comprises at least two markers from the first group and at least two markers from the second group.

4. The panel of claim 1, wherein the panel comprises at least three markers from each group.

5. The panel of claim 1, wherein the first group of markers comprises at least one of tryptophan, alpha-tocopherol, beta-tocopherol, gamma-tocopherol, gamma-CEHC, glycolithocholate sulfate, taurolithocholate 3-sulfate, biliverdin, bilirubin (Z,Z), bilirubin (E,E), 3-methyl-2-oxobutyrate, 3-methyl-2-oxovalerate, 4-methyl-2-oxopentanoate, isobutyrylcarnitine, 3-hydroxyisobutyrate, 2-methylbutyrylcarnitine, isovalerylcarnitine, propionylcarnitine, phenyllactate (PLA), phenylacetylglutamine, p-cresol sulfate, indolepropionate (IPA), 7-alpha-hydroxy-3-oxo-4-cholestenoate (7-Hoca), 3-indoxyl sulfate, cholesterol, and serum urea.

6. The panel of claim 1, wherein the second group of markers comprises at least one of 13-HODE, 9-HODE, leukotriene B4, 5-HETE, 5-oxoETE, 9(S)-hydroperoxy-10E,12Z-octadecadienoic acid (9(S)-HpODE), 5-HEPE, methionine sulfoxide, HWESASXX (SEQ ID NO:1), XHWESASXXR (SEQ ID NO:2), HWESASLLR (SEQ ID NO:3), HXGXA (SEQ ID NO:4), prostaglandin A2, prostaglandin E2, 7-alpha-hydroxycholesterol, 7-beta-hydroxycholesterol, a γ-glutamyl amino acid, and arginine.

7. The panel of claim 1, wherein the second group of markers further comprises at least one fatty acid.

8. The panel of claim 1, wherein the control comprises serum from one or more subjects who experienced term birth.

9. A kit comprising, the panel of claim 1, a first detection reagent specific for the first marker, and a second detection reagent specific for the second marker.

10. The kit of claim 9, wherein the first detection reagent and second detection each independently comprises at least one antibody.

11. A method of diagnosing or predicting the likelihood of occurrence of preterm birth (PTB) in a subject, the method comprising the steps of:
collecting a serum sample from a subject during the second trimester of pregnancy; and
measuring differences in levels of markers of the panel of claim 1 compared to a control,
wherein the levels of one or more of the markers are measured by means of one or more of antibody detection, chromatography, mass spectrometry, and a lipid test.

12. A kit for diagnosing or predicting the likelihood of occurrence of preterm birth (PTB) in a subject, the kit comprising:
a support;
a first marker from a first group of markers immobilized on the support;
a second marker from a second group of markers immobilized on the support; and
a detection reagent,
wherein a significant decrease in the first marker and a significant increase in the second marker measured in serum taken from a subject during the second trimester of pregnancy compared to a control is diagnostic or predictive of an increased risk of preterm birth for the subject.

13. The kit of claim 12, wherein the kit includes a marker from the first group and a marker from the second group.

14. The kit of claim 12, wherein the kit comprises at least two markers from the first group and at least two markers from the second group.

15. The kit of claim 12, wherein the kit comprises at least three markers from each group.

16. The kit of claim 12, wherein the first group of markers comprises at least one of tryptophan, alpha-tocopherol, beta-tocopherol, gamma-tocopherol, gamma-CEHC, glycolithocholate sulfate, taurolithocholate 3-sulfate, biliverdin, bilirubin (Z,Z), bilirubin (E,E), 3-methyl-2-oxobutyrate, 3-methyl-2-oxovalerate, 4-methyl-2-oxopentanoate, isobutyrylcarnitine, 3-hydroxyisobutyrate, 2-methylbutyrylcarnitine, isovalerylcarnitine, propionylcarnitine, phenyllactate (PLA), phenylacetylglutamine, p-cresol sulfate, indolepropionate (IPA), 7-alpha-hydroxy-3-oxo-4-cholestenoate (7-Hoca), 3-indoxyl sulfate, cholesterol, and serum urea.

17. The kit of claim 1, wherein the second group of markers comprises at least one of 13-HODE, 9-HODE, leukotriene B4, 5-HETE, 5-oxoETE, 9(S)-hydroperoxy-10E,12Z-octadecadienoic acid (9(S)-HpODE), 5-HEPE, methionine sulfoxide, HWESASXX (SEQ ID NO:1), XHWESASXXR (SEQ ID NO:2), HWESASLLR (SEQ ID NO:3), HXGXA (SEQ ID NO:4) prostaglandin A2, prostaglandin E2, 7-alpha-hydroxycholesterol, 7-beta-hydroxycholesterol, a γ-glutamyl amino acid, and arginine.

18. A method of determining the likelihood of preterm birth in a subject, the method comprising the steps of:
analyzing a sample taken from a subject to determine the levels of a first marker that when significantly lower in concentration compared to a term birth control is predictive of preterm birth and a second marker that when significantly higher in concentration compared to the term birth control is predictive of preterm birth; and
comparing the levels of the first and second markers to a term birth metabolic profile control in order to determine the likelihood of preterm birth in the subject.

19. The method of claim 18, wherein the levels of the first and second markers are measured by means of one or more of antibody detection, chromatography, mass spectrometry, and a lipid test.

20. The method of claim 18, wherein the sample is taken from a subject during the second trimester of pregnancy.
